(19) United States
(12) Patent Application Publication
(10) Pub. No.: US 2005/0215485 A1
(43) Pub. Date: Sep. 29, 2005

(54) Liquid preparation comprising camptothecin derivative and pharmaceutical composition producible by lyophilizing the preparation

(76) Inventors: Takahiro Ito, Hyogo-ken (JP); Shinji Narisawa, Osaka-fu (JP)

Correspondence Address: BIRCH STEWART KOLASCH & BIRCH, PO BOX 747, FALLS CHURCH, VA, 22040-0747, US

(21) Appl. No.: 10/509,912

(22) PCT Filed: Apr. 15, 2003
(86) PCT No.: PCT/JP03/04745

§ 371 Date: Oct. 4, 2004

(30) Foreign Application Priority Data
Apr. 16, 2002 (JP) 2002-112864

(51) Int. Cl. A61K 38/06; A61K 38/05; C07K 5/06

(52) U.S. Cl. 514/18; 530/330; 530/331

(57) ABSTRACT

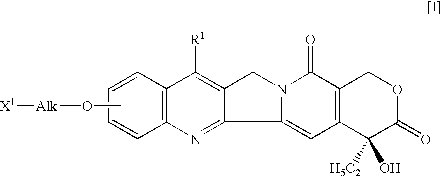

The present invention relates to a stable liquid preparation comprising a camptothecin derivative which is prepared by binding a compound of the formula [I]: wherein R₁ is a substituted or unsubstituted lower alkyl group, X₁ is a group of the formula: —NHR₂ (R₂ is a hydrogen atom or a lower alkyl group) or a hydroxy group and Alk is a straight or branched chain alkylene group optionally interrupted by an oxygen atom, and a polysaccharide having carboxyl groups via an amino acid or a peptide, or a pharmaceutically acceptable sat thereof, which is adjusted to pH 5-8, or a stable pharmaceutical composition produced by lyophilizing said liquid preparation.

### TECHNICAL FIELD

The present invention relates to a liquid preparation comprising a camptothecin derivative or a pharmaceutically acceptable salt thereof, which shows excellent antitumor activities, a pharmaceutical composition that is producible by lyophilizing said liquid preparation, and a process for preparing said pharmaceutical composition.

More particularly, the present invention relates to a liquid preparation for injection comprising a camptothecin derivative which is prepared by binding a compound of the formula [I]: 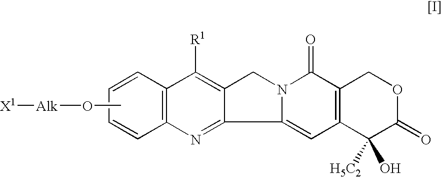
wherein R¹ is a substituted or unsubstituted lower alkyl group, X¹ is a group of the formula: —NHR² (R² is a hydrogen atom or a lower alkyl group) or a hydroxy group and Alk is a straight or branched chain alkylene group optionally interrupted by an oxygen atom, and a polysaccharide having carboxyl groups via an amino acid or a peptide, or a pharmaceutically acceptable salt thereof, which is adjusted to pH 5-8, or a pharmaceutical composition produced by lyophilizing said liquid preparation, or a process for preparing the same.

### BACKGROUND ART

The camptothecin derivatives of the present invention and pharmaceutically acceptable salts thereof are medicinal substances that show excellent antitumor activities against various tumors, especially they show excellent therapeutic effects on solid tumors such as pulmonary cancer, uterine cancer, ovarian cancer, breast cancer, or gastrointestinal cancer (large bowel cancer, gastric cancer, etc.). It has been known that said compounds can be administered parenterally (e.g. intravascular injection) generally in the form of a liquid preparation (e.g. solution, suspension, emulsion, etc.) (JP-10-72467A, EP-0757049A)

### DISCLOSURE OF INVENTION

The camptothecin derivative above has the structure wherein a camptothecin compound (active substance) of the formula [I] is bound to a polysaccharide (carboxymethylated dextran or pullulan) through a spacer (an amino acid or a peptide). Said camptothecin derivatives, when formulated into a liquid preparation, often undergo hydrolysis at the site of spacer or polysaccharide moiety during the preparation process or storage. Hydrolysis of the polysaccharide moiety results in the reduction of the mean molecular weight of said camptothecin derivatives and the increase of the molecular weight distribution, which variation of molecular weight is apt to affect adversely to the pharmacokinetics of said medicinal substance. Further, hydrolysis of the spacer would result in the release of a considerable amount of an active substance (camptothecin compound [I]) at the time of preparation, which is unfavorable in terms of therapeutic effects or side effects. Accordingly, it was desired to find a liquid preparation that is excellent as to drug stability during the preparation process and storage.

The present inventors have intensively studied to solve the problems above, and have found that a liquid preparation with excellent stability can be obtained by adjusting the pH of a liquid preparation comprising a camptothecin derivative of the present invention between 5 and 8 during the preparation process thereof, and have accomplished the present invention.

That is, the present invention provides a liquid preparation for injection comprising a camptothecin derivative wherein a camptothecin compound of the formula [I] above is bound to a polysaccharide having carboxyl groups via an amino acid or a peptide, or a pharmaceutically acceptable salt thereof, which preparation is adjusted to pH 5-8.

Further, the present inventors have found that a pharmaceutical composition prepared by lyophilizing the liquid preparation above also shows excellent drug stability during the preparation process and storage. Accordingly, the present invention also provides such a pharmaceutical composition.

### MODE FOR CARRYING OUT THE INVENTION

In the present invention, any one(s) of camptothecin derivatives disclosed in JP-10-72467A, that is, camptothecin derivatives wherein a camptothecin compound of the formula [I] above is bound to a polysaccharide having carboxyl groups via an amino acid or a peptide can be used. Specific examples of the camptothecin derivatives include those wherein X¹ of a compound [I] and a carboxyl group of an amino acid or a peptide (e.g. a peptide consisting of 2-5 amino acids) are bound to form an acid-amide bond or an ester bond, and an amino group of said amino acid or peptide and a part or all carboxyl groups of a polysaccharide such as a carboxymethylated dextran or pullulan are bound to form an acid-amide bond(s).

More specifically camptothecin derivatives include those in which a part or all carboxyl groups of a polysaccharide are bound to a N-terminal amino group of an amino acid or a peptide to form an acid-amide bond, and a C-terminal carboxyl group of said amino acid or peptide is bound with X¹ of a compound of [I] to form an acid-amide bond or an ester bond.

Substituents on a compound of a generic formula [I] include the following substituents. When X² is —NHR², a lower alkyl group in R² includes a C₁₋₄ alkyl group, and a substituent on a lower alkyl group in R¹ includes a hydroxy group optionally protected, a mercapt group and an amino group (e.g. optionally protected by an alkyl group or an acyl group) Alk includes a straight or branched chain C₁₋₆ alkylene group which is optionally interrupted by an oxygen atom.

Polysaccharides related to the present invention include a polysaccharide having originally a carboxyl group in its molecule (e.g. hyaluronic acid, pectin, etc.), a polysaccharide (e.g. carboxymethylated pullulan, carboxymethylated dextran, etc.) which is prepared by introducing a carboxyl group into a polysaccharide having originally no carboxyl group in its molecule (e.g. pullulan, dextran, etc.). Among them carboxymethylated dextran (e.g. degree of carboxymethylation is more than 0.3 and less than 0.8) is especially preferable. Its mean molecular weight is preferably 20,000-400,000, especially preferably 50,000-150,000.

Preferable camptothecin derivatives are those wherein R¹ is an unsubstituted C₁₋₆ alkyl group, X¹ is an amino group and Alk is a straight chain C₁₋₆ alkylene group not interrupted by an oxygen atom, a polysaccharide is a carboxymethylated dextran or pullulan, and a peptide is a peptide consisting of 2-5 amino acids.

More preferable camptothecin derivatives are those wherein R¹ is ethyl group, a group of the formula: X¹-Alk-O— is 3-aminopropyloxy group, and camptothecin compound [I] bound at position 10 of a camptothecin nucleus and dextran in which a carboxyl group is introduced, are bound via a peptide selected from a group consisting of glycyl-glycyl-L- or D-phenylalanyl-glycine, glycyl-glycine, glycyl-glycyl-glycine, glycyl-glycyl-glycyl-glycine, glycyl-glycyl-glycyl-glycyl-glycine, L- or D-phenylalanyl-glycine, and L- or D-leucyl-glycine. Among those peptides, glycyl-glycyl-glycine is especially preferable.

As pharmaceutically acceptable salts of camptothecin derivatives, alkali metal salts such as sodium salt or potassium salt, alkaline earth metal salts such as calcium salt, or amino acid salts such as arginine salt or lysine salt are illustrated.

The liquid preparation of the present invention is prepared, for example as follows; (1) a camptothecin derivative above or its pharmaceutically acceptable salt and if necessary other ingredients (e.g. excipients for the pharmaceutical preparations such as buffer, a stabilizing agents) are dissolved in a liquid medium such as water for injection etc., (2) the solution is adjusted to pH 5-8, preferably 5-7.5, more preferably 5-7, especially preferably 6-7 with a suitable buffer (e.g. citric acid, hydrochloric acid, sodium hydroxide, etc.), and then, (3) after diluted with water for injection to get desired drug concentration, the solution is filtered through a membrane filter etc., to remove the insoluble materials (pyrogen etc.) and then is filled into a sealing grass vessel, followed by sterilization to prepare the liquid preparation.

The amount of a camptothecin derivative or a pharmaceutically acceptable salt thereof is not limited, but is 1% (w/v) to 20% (w/v), preferably 1% (w/v) to 10% (w/v).

Buffer used for the liquid preparation of the present invention is selected from the group consisting of citric acid, an alkali metal citrate (e.g. sodium citrate etc.), acetic acid, an alkali metal acetate (e.g. sodium acetate etc.), and an alkali metal dihydrogen phosphate (sodium dihydrogen phosphate etc.). These compounds are suitably combined to use as the buffer. The preferable combination as the buffer is a combination of citric acid and sodium citrate, a combination of citric acid and sodium dihydrogen phosphate, and a combination of acetic acid and sodium acetate, preferably a combination of citric acid and sodium citrate. Ionic strength of the buffer used for the liquid preparation of the present invention can be adjusted to, for example, 0.01-0.6, preferably 0.01-0.3, especially preferably 0.05-0.2.

To the liquid preparation of the present invention and the lyophilized composition thereof can be added conventional ingredients used for injection as well as the above mentioned ingredients. These ingredients are fillers (lactose, sucrose, mannitol, dextran, maltose, trehalose, etc.), solubilizing agents (polyoxyethylene solbitan fatty acid ester such as polysolbate 80 etc., polyoxyethylene hydrogenated castor oil such as HCO-60 etc, polyoxyethylene alkyl ether such as polyoxyethylene lauryl ether, solbitan fatty acid ester such as Span 80 etc.), stabilizer (alkali metal carbonate such as sodium carbonate, alkali hydrogen carbonate such as sodium hydrogen carbonate etc.), antioxidants (cysteine hydrochloride, tocopherol, ascorbic acid, etc.), tonicity agents (glycerin, glucose, etc.), and preservatives (thimerosal, ethanol, propylene glycol, benzyl alcohol, para hydoxybenzoic acid alkyl ester such as para hydoxybenzoic acid butyl ester, etc.).

The amount of the filler is, for example, 10-100% to a camptothecin derivative [I] or a pharmaceutically acceptable salt thereof. The amount of the solubilizer is, for example, 0.1-10% to a camptothecin derivative [I] or a pharmaceutically acceptable salt thereof. The amount of the stabilizer is, for example, 0.1-10% to a camptothecin derivative [I] or a pharmaceutically acceptable salt thereof. The amount of the antioxidant is, for example, 0.1-10% to a camptothecin derivative [I] or a pharmaceutically acceptable salt thereof. The amount of the tonicity agent is for example, 0.01-1% to a camptothecin derivative [I] or a pharmaceutically acceptable salt thereof. The amount of the preservative is, for example, 0.001-0.2% to a camptothecin derivative [I] or a pharmaceutically acceptable salt thereof.

The liquid preparation prepared above is filled into a hard vessel such as a sterile ampoule, a vial, a syringe, etc., and is lyophilized by a conventional method to prepare the pharmaceutical composition of the present invention.

The lyophilized pharmaceutical composition of the present invention is prepared as follows.

The amount of the liquid preparation to be filled into a vessel is, for example, preferably 5-50% (v/v) per the volume of the vessel, especially preferably 10-25% (v/v).

The external temperature on lyophilization is kept preferably at −50 to 60° C., especially preferably −50 to 40° C., and the pressure for sublimation of the solvent used is preferably 0.01-0.2 Torr, more preferably 0.01-0.1 Torr. The rate of lyophilization is preferably adjusted such that the volume of the solvent (calculated into a solution) is sublimated at the rate of 10 μl to 100 μl per 1 cm² of the surface area from which the solvent is sublimated for one hour, especially 30 μl to 60 μl under controlling ingredients of the liquid to be lyophilized, temperature at lyophilization, pressure at sublimation of the solvent, etc.

In case of lyophilizing the liquid preparation, especially the preparation containing mannitol, dextran, and/or sodium carbonate, etc., the breakage of the vessel is protected by previously adding at least one salt selected from the group consisting of alkali metal chlorides (lithium chloride, sodium chloride, potassium chloride, etc.), alkaline earth metal chlorides (magnesium chloride, calcium chloride, etc.) and alkali metal sulfates (lithium sulfate, potassium sulfate, sodium sulfate, etc.), to said liquid preparation. In this case, preferable salts are sodium chloride, sodium sulfate, etc. The amount of said salt is preferably 0.01-10%, more preferably 0.1-5% per the drug (weight).

The liquid preparation and the pharmaceutical composition prepared by lyophilizing the liquid preparation are preferably stored in a light resistant sealing vessel.

The liquid preparation of the present invention as prepared above, has an excellent property as to drug-stability (a camptothecin derivative) during the preparation process or storage. Therefore, the liquid preparation can be administered directly to a patient. The dosage of the liquid preparation is varied on age, body weight, or condition, but is usually 0.02-50 mg, especially 0.1-10 mg/kg in calculation to a camptothecin compound [I] (in case of X¹ being —NHR², its hydrochloride).

The pharmaceutical composition prepared by lyophilizing the liquid preparation of the present invention, has also an excellent property as to drug-stability during the preparation process or storage, and therefore, it is useful for an injection prepared when necessary.

The present invention is further explained in detail by examples, but the present invention should not be limited by these examples.

### EXAMPLE 1

Preparation for Liquid Preparations

Based on ingredients of Table 1 below, an aqueous drug solution was prepared and filtered through a membrane filter (type: GS, pore diameter: 0.22 μm prepared by Millipore Ltd.). The filtrate (1 mL) was filled into a grass 3 mL-ampoule. Each ampoule was sterilized in vapor at 100° C. for 15 min to obtain a liquid preparation. Drug: Camptothecin derivative described in Example 84 of Jp-10-72467A as represented by the following formula:

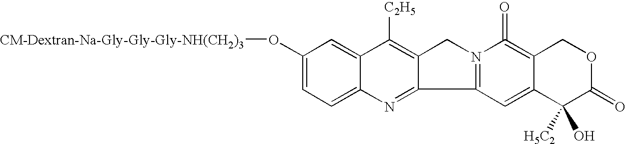

wherein CM means “carboxymethylated”.

TABLE 1

| | | Liq. preparation of | | | |
|---|---|---|---|---|---|
| | Comparative | present invention | | | |
| | example | 1 | 2 | 3 | 4 |
| Drug (g) | 0.4 | | | | |
| Sodium dihydrogen | 0.110 | 0.147 | 0.180 | 0.213 | 0.245 |
| phosphate(g) | | | | | |
| Citric acid | 0.118 | 0.093 | 0.071 | 0.047 | 0.023 |
| 0.4M Aq. sodium | q.s. | q.s. | | | |
| dihydrogen | | | | | |
| phosphate solution | | | | | |
| 0.2M Aq. citric | q.s. | q.s. | | | |
| acid solution | | | | | |
| Sodium chloride(g) | 0.771 | 0.771 | | | |
| Water for injection | q.s. | q.s. | | | |
| Total | 100 mL | 100 mL | | | |
| pH | 4.0 | 5.0 | 6.0 | 7.0 | 8.0 |

Stability of Liquid Preparation

The preparation prepared above was preserved under each preservation condition (at 60° C. for 20 days, 50° C. for 30 days or 40° C. for 120 days), and the stability of the drug was tested (Mean molecular weight and molecular weight distribution, and amount of free active camptothecin). The result was shown in the following Table 2. The mean molecular weight of the drug was calculated by GPC multi angles Laser scattering method (MALLS method) and the mean molecular weight distribution was calculated by the following formula:
Mean molecular weight distribution=weight of mean molecular weight (MW)/number of mean molecular weight (MN)

TABLE 2

| | | | | Mean |
|---|---|---|---|---|
| | | | Mean | molecular |
| | | Preservation | molecular | weight |
| | pH | condition | weight | distribution |
| Liq. | 5.0 | Initial | 138,900 | 1.195 |
| preparation 1 | | 60° C. for | 125,800 | 1.183 |
| of present | | 20 days | | |
| invention | | | | |
| Liq. | 6.0 | Initial | 129,100 | 1.169 |
| preparation 2 | | 60° C. for | 131,200 | 1.177 |
| of Present | | 20 days | | |
| invention | | | | |
| Liq. | 7.0 | Initial | 131,400 | 1.191 |
| preparation 3 | | 60° C. for | 131,400 | 1.186 |
| of Present | | 20 days | | |
| invention | | | | |
| Liq. | 8.0 | Initial | 130,900 | 1.202 |
| preparation 4 | | 60° C. for | 127,000 | 1.195 |
| of Present | | 20 days | | |
| invention | | | | |
| Comparative | 4.0 | Initial | 129,800 | 1.200 |
| example | | 60° C. for | 110,100 | 1.720 |
| | | 20 days | | |

TABLE 3

| | | Amount of free active camptothecin | | | |
|---|---|---|---|---|---|
| | | compound (%)* | | | |
| | | | 60° C. for | 50° C. for | 40° C. for |
| | pH | Initial | 20 days | 30 days | 120 days |
| Liq. | 5.0 | 3.08 | 13.15 | 9.17 | 10.60 |
| preparation 1 | | | | | |
| of present | | | | | |
| invention | | | | | |
| Liq. | 6.0 | 1.67 | 8.12 | 5.83 | 5.53 |
| preparation 2 | | | | | |
| of present | | | | | |
| invention | | | | | |
| Liq. | 7.0 | 1.48 | 14.53 | 6.60 | 6.52 |
| preparation 3 | | | | | |
| of present | | | | | |
| invention | | | | | |
| Liq. | 8.0 | 1.93 | 17.32 | 8.31 | 7.95 |
| preparation 4 | | | | | |
| of present | | | | | |
| invention | | | | | |
| Comparative | 4.0 | 13.81 | 23.73 | 24.84 | 27.33 |
| example | | | | | |

*Active camptothecin compound means a compound of the following formula and the amount was quantitatively analyzed by the following conditions (the same hereinafter).

Quantitative analysis: A sample solution was diluted with 0.2M formic acid-ammonium formate buffer in 200 times and then, the diluted solution (0.4 mL) and an internal standard solution (0.1 mL) were mixed and the mixture was filtered through a membrane filter (pore diameter; 0.45 μm) to prepare a test sample for quantitative analysis. The sample was quantitatively analyzed by subjecting to HPLC under the following conditions.

The amount (%) of free active camptothecin in each sample was calculated as 100% of the amount of free active camptothecin compound produced by adding 10 times amount of 6N hydrochloric acid to the sample solution preserved in a refrigerator and then heating at 100° C. for 4 hours. HPLC conditions:
Column: Inertsil ODS (prepared by GL Science Inc.)
Mobile phase: 35 mM formic acid-ammonium formate buffer (pH3)/acetonitrile=80/20 (flow: 1.0 mL/min.)
Column temperature: 40° C.
Detection: Fluorophotometer (Ex=360, Em=420 nm)
Active camptothecin compound: 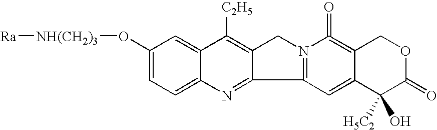
wherein Ra is hydrogen atom, Gly-, Gly-Gly- or Gly-Gly-Gly-.

From the result above, in the liquid preparations of the present invention (pH 5-8), decrease of the mean molecular weight of the drug is less in comparison with a liquid preparation of the comparative example and therefore, increase of the molecular weight distribution of the drug was recognized being protected. This reveals that in the liquid preparation of the present invention degradation of the drug (namely cleavage of chain of dextran molecule) can be prevented and undesired formation of free active camptothecin compound due to degradation of spacer portion can be also prevented.

### EXAMPLE 2

Preparation of Lyophilized Compositions

Using the same drug as the drug of Example 1, and based on ingredients described in Table 4 each aqueous drug solution was prepared and filtered through a membrane filter (type: GS, pore diameter: 0.22 μm prepared by Milipore Ltd.). The filtrate (1 mL), was filled into a colorless 13-mL vial and the vial was sealed. Each vial was subjected to lyophilization (pre-freezing: −50° C. for 3 hours, primary dehydration: 20° C. for 30 hours, secondary dehydration: 60° C. for 6 hours) to prepare a lyophilized drug composition.

TABLE 4

| | Comparative | | Composition of present | | | |
|---|---|---|---|---|---|---|
| | example | | invention | | | |
| | A | B | 1 | 2 | 3 | 4 |
| Drug (g) | 5.0 | | | | | |
| Sodium | 0.0059 | 0.110 | 0.147 | 0.180 | 0.213 | 0.245 |
| dihydrogen | | | | | | |
| phosphate (g) | | | | | | |
| Citric acid | 0.153 | 0.118 | 0.093 | 0.071 | 0.047 | 0.023 |
| 0.4M Aq. Sodium | q.s. | | q. s. | | | |
| dihydrogen | | | | | | |
| phosphate | | | | | | |
| solution | | | | | | |
| 0.2M Citric acid | q.s. | | q.s. | | | |
| solution | | | | | | |
| Water for | q.s. | | q.s. | | | |
| injection | | | | | | |
| Total | 100 mL | | 100 mL | | | |
| pH | 3.0 | 4.0 | 5.0 | 6.0 | 7.0 | 8.0 |

Stability of Lyophilized Compositions

The preparations prepared above were preserved at 60° C. for 20 days, and the stability of the drug compositions was tested (Change of color, Insoluble materials are present or not after reconstitution, molecular weight distribution of the drug, and amount of free active compound). The result was shown in the following Tables 5-1 and 5-2.

TABLE 5-1
[presence of insoluble materials or not]

| | Comparative | | Composition of | | | |
|---|---|---|---|---|---|---|
| | example | | present invention | | | |
| | A | B | 1 | 2 | 3 | 4 |
| Change of | No | No (pale | No (pale yellowish | | | |
| color | (yellow) | yellowish | green) | | | |
| | | green) | | | | |
| State after | Insoluble | Insoluble | Insoluble material: no | | | |
| reconstitution | material: | material: | | | | |
| | yes | slight | | | | |
| pH after | 3.0 | 4.1 | 5.1 | 6.1 | 7.1 | 8.1 |
| reconstitution | | | | | | |

TABLE 5-2
[Change of mean molecular weight and molecular weight
distribution of drug]

| | | | | Mean |
|---|---|---|---|---|
| | | | Mean | molecular |
| | | Condition of | molecular | weight |
| | pH | preservation | weight | distribution |
| Composition | 5.0 | Initial | 135,400 | 1.144 |
| of present | | 60° C. for 20 days | 128,700 | 1.145 |
| invention 1 | | | | |
| Composition | 6.0 | Initial | 132,800 | 1.145 |
| of present | | 60° C. for 20 days | 128,500 | 1.140 |
| invention 2 | | | | |
| Composition | 7.0 | Initial | 130,600 | 1.143 |
| of present | | 60° C. for 20 days | 128,300 | 1.147 |
| invention 3 | | | | |
| Composition | 8.0 | Initial | 129,800 | 1.144 |
| of present | | 60° C. for 20 days | 131,100 | 1.128 |
| invention 4 | | | | |
| Comparative | 3.0 | Initial | 132,900 | 1.120 |
| example A | | 60° C. for 20 days | 150,300 | 1.280 |
| Comparative | 4.0 | Initial | 135,100 | 1.134 |
| example B | | 60° C. for 20 days | 138,100 | 1.209 |

TABLE 5-3
[Amount of free active compound]

| | | Amount of free active compound (%) |
|---|---|---|
| | pH | (Preserved conditions: 60° C. for 20 days) |
| Composition 1 | 5.0 | <0.3 |
| of present | | |
| invention | | |
| Composition 2 | 6.0 | <0.3 |
| of present | | |
| invention | | |
| Composition 3 | 7.0 | <0.3 |
| of present | | |
| invention | | |
| Composition 4 | 8.0 | <0.3 |
| of present | | |
| invention | | |
| Comparative | 3.0 | 0.76 |
| example A | | |
| Comparative | 4.0 | 0.48 |
| example B | | |

### EXAMPLE 3

Preparation of Lyophilized Compositions

The same drug as example 1 (10 g), citric acid monohydrate (0.42 g), and sodium chloride (500 mg) were dissolved in water for injection (100 mL) and the solution was adjusted to pH 5.0 with 1M sodium hydroxide to make the total volume 200 mL by adding water for injection. The solution was filtered through a membrane filter (type: GS, pore diameter: 0.22 μm prepared by Milipore Ltd.) and the filtrate (2 ml), was filled into a colorless grass 3-mL ampoule. Each ampoule was lyophilized by a usual method to prepare a lyophilized preparations prepared when necessary (the preparation of the present invention).

As a comparative example, the same drug (10 g) as used in example 1, and citric acid monohydrate (0.42 g) were dissolved in water for injection (100 mL) and the solution was treated by the same manner as mentioned above to prepare lyophilized preparations prepared when necessary (Sodium chloride was not added.).

The breakage of the grass ampoules was tested on the composition of the present invention and the composition of the comparative example. The result was shown in the following Table 6.

TABLE 6

| | Broken number |
|---|---|
| | per 100 ampoules |
| Lyophilized composition of | 0 |
| the present invention | |
| Lyophilized composition of | 40 |
| Comparative example | |

### EXAMPLE 4

Preparation of Lyophilized Compositions

The same drug as example 1 (5 g), citric acid monohydrate (0.093 g), anhydrous sodium dihydrogen phosphate (0.147) and sodium chloride (50 mg) are dissolved in water for injection (50 mL) and the solution is adjusted to pH 5.0 with 0.4M aqueous sodium dihydrogen phosphate solution or 0.2M aqueous citric acid solution to make the total volume 100 mL by adding water for injection. The solution is filtered through a membrane filter (type: GS, pore diameter: 0.22 μm prepared by Milipore Ltd.) and the filtrate (20 ml) is filled into a grass 100-mL vial. Each vial is lyophilized by a usual method to prepare lyophilized compositions prepared when necessary.

### EXAMPLE 5

Preparation of Lyophilized Compositions

The same drug as example 1 (5 g), citric acid monohydrate (0.093 g), sucrose (5 g) and sodium chloride (50 mg) are dissolved in water for injection (50 mL) and the solution is adjusted to pH 6.0 with 1M aqueous sodium hydroxide solution to make the total volume 100 mL by adding water for injection. The solution is filtered through a membrane filter (type: GS, pore diameter: 0.22 μm prepared by Milipore Ltd.) and the filtrate (20 ml) is filled into a grass 100 mL-vial. Each vial is lyophilized by a usual method to prepare lyophilized composition prepared when necessary.

### EFFECT OF THE INVENTION

The liquid preparation of the present invention and the composition prepared by its lyophilization have an excellent effect that the degradation of the drug (camptothecin) is less in any stage such as its preparation process, distribution and preservation.

1. A liquid preparation comprising a camptothecin derivative which is prepared by binding a compound of the formula [I]: 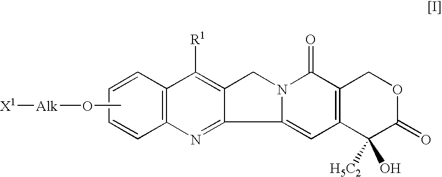
wherein R¹ is a substituted or unsubstituted lower alkyl group, X¹ is a group of the formula: —NHR² (R² is a hydrogen atom or a lower alkyl group) or a hydroxy group and Alk is a straight or branched chain alkylene group optionally interrupted by an oxygen atom, and a polysaccharide having carboxyl groups via an amino acid or a peptide, or a pharmaceutically acceptable salt thereof, which is adjusted to pH 5-8.

2. The liquid preparation according to claim 1 wherein one or more compounds selected from the group consisting of citric acid, an alkali metal citrate, acetic acid, an alkali metal acetate and an alkali metal dihydrogen phosphate are used as the buffer.

3. The liquid preparation according to claim 2 wherein ionic strength of the buffer is 0.2 or less than 0.2.

4. The liquid preparation according to any one of claims 1 to 3 wherein the pH is adjusted to 5 to 7.5.

5. The liquid preparation according to any one of claims 1 to 3 wherein the pH is adjusted to 5 to 7.

6. The liquid preparation according to any of claims 1 to 3 wherein the pH is adjusted to 6 to 7.

7. The liquid preparation according to any one of claims 1 to 6 wherein the amount of the camptothecin derivative or its pharmaceutically acceptable salt is 1% to 20%.

8. The liquid preparation according to claim 1, wherein one ore more ingredients selected from a stabilizer and a filler are further contained.

9. The liquid preparation according to claim 1, wherein one or more stabilizers selected from an alkali metal carbonate and alkali metal hydrogen carbonate, and one ore more fillers selected from lactose, sucrose, mannitol, dextran, maltose and trehalose are further contained.

10. The liquid preparation according to claim 1, wherein one or more salts selected from an alkali metal chloride, an alkaline earth metal chloride and an alkali metal sulphate are further contained.

11. The liquid preparation according to claim 1 wherein R¹ is an unsubstituted C₁₋₆ alkyl group, X¹ is an amino group and Alk is a straight chain C₁₋₆ alkylene group not interrupted by an oxygen atom, a polysaccharide is a carboxymethylated dextran or pullulan, and a peptide is a peptide consisting of 2-5 amino acids.

12. The liquid preparation according to claim 11 wherein R¹ is ethyl group, a group of the formula: X¹-Alk-O— is 3-aminopropyloxy group, and the camptothecin compound [I] is bound at position 10 of a camptothecin nucleus, the polysaccharide is dextran in which a carboxyl group is introduced, the peptide is glycyl-glycyl-L- or D-phenylalanyl-glycine, glycyl-glycine, glycyl-glycyl-glycine, glycyl-glycyl-glycyl-glycine (SEQ ID NO: 1), glycyl-glycyl-glycyl-glycyl-glycine (SEQ ID NO: 2), L- or D-phenylalanyl-glycine, and L- or D-leucyl-glycine.

13. The liquid preparation according to claim 12 wherein the peptide is glycyl-glycyl-glycine.

14. A lyophilized drug composition prepared by lyophilizing the liquid preparation according to claim 1.

15. A liquid composition for injection wherein the composition according to claim 14 is dissolved in an aqueous medium.

16. A liquid preparation comprising a camptothesin derivative, which is prepared by binding a compound of the formula (Ia): 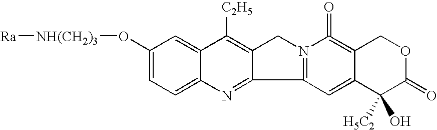
and a dextran having carboxylic groups via glycil-glycil-glycil, or a pharmaceutically acceptable salt thereof, wherein the liquid preparation is adjusted to pH 5 to 8 with a buffer.

17. The liquid preparation according to claim 16, wherein the buffer is one or more compounds selected from the ciric acid, an alkali metal citrate, acetic acid, an alkali metal acetate and an alkali metal dihydrogen phosphate.

18. The liquid preparation according to claim 17, wherein the buffer is citric acid and sodium dihydrogen phosphate.

19. The liquid preparation according to claim 18, wherein sodium chloride is further contained.


SEQUENCE LISTING (2 sequences)

SEQ ID NO: 1 - protein, 4 aa, Artificial sequence
  synthetic peptide spacer existing between a polysaccharide having a carboxyl group and an active substance having anti-tumor activity
GGGG

SEQ ID NO: 2 - protein, 5 aa, Artificial sequence
  synthetic peptide spacer existing between a polysaccharide having a carboxyl group and an active substance having an anti-tumor activity
GGGGG